(19) United States
(12) Patent Application Publication
(10) Pub. No.: US 2018/0008551 A1
(43) Pub. Date: Jan. 11, 2018

(54) METALLIC AND PROTEIN/POLYMER NANOPARTICLE CONSTRUCTS, MULTI-DRUG VEHICLE AND FABRICATION METHODS

(71) Applicant: The Curators of the University of Missouri, Columbia, MO (US)

(72) Inventors: Raghuraman Kannan, Columbia, MO (US); Srikar Raman, Columbia, MO (US); Ajit Zambre, Columbia, MO (US); Abilash Gangula, Columbia, MO (US); Anandhi Upendran, Columbia, MO (US)

(21) Appl. No.: 15/643,094

(22) Filed: Jul. 6, 2017

Related U.S. Application Data
(60) Provisional application No. 62/359,513, filed on Jul. 7, 2016.

(51) Int. Cl. A61K 9/51; A61K 31/704; A61K 31/7068; A61K 33/24

(52) U.S. Cl. CPC A61K 9/5169; A61K 33/24; A61K 9/5115; A61K 31/704; A61K 31/7068

(57) ABSTRACT

A multidimensional nanoconstruct includes a three-dimensional thiolated protein, gelatin or polymer nanoparticle and exposed metallic nanoparticles bonded to outer surfaces of the particle. In a method of formation, number of metallic nanoparticles that attach to the carrier nanoparticle is controlled via microfluidics or via controlling the reactivity of the metallic nanoparticle and carrier nanoparticle.

### PRIORITY CLAIM AND REFERENCE TO RELATED APPLICATION

The application claims priority under 35 U.S.C. §119 and all applicable statutes and treaties from prior U.S. provisional application Ser. No. 62/359,513, which was filed Jul. 7, 2016.

### FIELD

A field of the invention is nanomaterials, and particularly multi-dimensional nanoconstructs. Example applications of the invention include in vivo treatment and sensing applications.

### BACKGROUND

Gold nanoparticles have a wide range of known applicability. Example uses include drug delivery, therapeutic uses, theranostic uses, sensors, photodynamic therapy, probes and catalysis. Physicochemical properties limit the wider applicability of the gold nanoparticles. Silver and other metallic nanoparticles similarly have many applications, which are limited by metal nanoparticle physicochemical properties.

Cytoplasmic delivery is advantageous in drug delivery. Nanoparticles fail endosomal escape because the particles tend to accumulate within endosome/lysosome upon cellular internalization. This is true of all nanoparticles known to the inventors that have been tested for drug delivery. Gold and other nanoparticles are therefore a poor choice for cytoplasmic delivery. Some methods of manufacturing gold nanoparticles render the particles a poor choice for this and other in vivo uses. Toxicity is associated with some surfactants, e.g. sodium borohydride) required for the preparation via this techniques (such as sodium borohydride reduced gold nanoparticles).

Sensing applications using gold nanoparticles, such as colorimetric sensors, realize a low sensitivity and narrow range of detection due. The performance limits can be attributed to non-directive cross-linking of the nanoparticles. Another cause is the fact that suspensions of gold nanoparticles are often unstable.

Others have attempted to form multi-dimensional nanoconstructs using gold nanoparticles and polymers. Such formation has proven synthetically challenging. Conventional bulk reactions by simple addition of gold nanoparticles to polymer suspensions produces wide variations and unpredictable nanoconstructs. Some have used the constructs for treatments, ranging from pharmacological activity to tissue engineering.

Prestwich et al U.S. Published Application No. 20110280914 discloses nanocomposites of a thiolated macro-molecule that crosslinks with gold nanoparticles to produce a composite that is useful in anchoring cells. The composites can be used to form multi-layer 3-D structures, where the cells in each layer can aggregate and fuse with one another to form tissues and organs. The constructs are formed by admixing one or more thiolated macromolecules with the gold nanoparticles in water.

Amiji et al PCT Published Application WO/2014/110578 discloses targeted magnetic nanoparticles fabricated using surface-functionalized super paramagnetic nanoparticles that are encapsulated in a biodegradable and biocompatible polymer to form microparticles. The microparticles are rendered target-selective by additional coatings of gelatin and gold nanoparticles which are derivatized with a targeting ligand specific for a targeted cell type. The particles are used to capture specific cell types, such as cancer circulating tumor cells. The particles are formed by embedding the nanoparticles of (a) in a matrix of hydrophobic polymer to form microparticles containing the nanoparticles; (c) coating the microparticles from (b) with a biopolymer to form a shell of the biopolymer surrounding the microparticles; and (d) attaching a plurality of targeting nanoparticles to the biopolymer shell of (c) to form the plurality of the releasable magnetic nanoparticle, wherein the targeting nanoparticles contain a plurality of targeting moieties attached to their surface

Microfluidics has been used to form drug encapsulated polymeric particles. See, Karnik R, Gu F, Basto P, Cannizzaro C, Dean L, Kyei-Manu W, Langer R, Farokhzad O C, “Microfluidic platform for controlled synthesis of polymeric nanoparticles.” Nano Lett 2008, 8:2906-2912. That method involved mixing and nanoprecipitation of polymers and drugs dissolved in organic solvents with nonsolvents. Hydrodynamic flow focusing in microfluidic channels was used to control nanoprecipitation of poly(lactic-co-glycolic acid)-b-poly(ethylene glycol) diblock copolymers. Varying flow rates, polymer composition, and polymer concentration was used to control particle size, improve polydispersity, and control drug loading and release of the resulting nanoparticles.

Much research has been devoted to the delivery of drugs and other payloads in vivo via nanoparticles and nanoconstructs. However, in the case of cancer cells, it has been shown that several enzymes such as cysteine proteases, serine proteases, aspartate proteases, threonine proteases and/or matrix metalloproteases (MMP) are upregulated. See, Rakashanda S, et al., “Role of proteases in cancer: a review,” Biotechnol. Mol. Biol. Rev. 2012, 7:90-101. Upregulation of gelatinase MMP-2 in various cancers is well documented and others have shown cytosolic enzymatic activity of the gelatinase MMP-2. See, Rollin J, et al., “Influence of MMP-2 and MMP-9 promoter polymorphisms on gene expression and clinical outcome of non-small cell lung cancer,” Lung Cancer 2007, 56:273-280; Ali M A M, et al. “Mechanisms of cytosolic targeting of matrix metalloproteinase-2. J. Cell. Physiol. 2012, 227:3397-3404. This makes cytoplasmic delivery an important goal, but gold nanoparticles fail cytoplasmic delivery as discussed above.

Gobbo et al. reported a gold nanoparticle and carbon nanotube hybrid. “Facile synthesis of gold nanoparticle (AuNP)—carbon nanotube (CNT) hybrids through an interfacial Michael addition reaction,” Chem. Commun., 2013, 49, 2831-2833. The synthesis method utilized a Michael addition reaction between thiol-functionalized single-wall CNT and small water-soluble Maleimide—AuNP. The authors explained the hybrid as resulting from a covalent bond linking the nanoparticle to the CNT and by a functionalization reaction of an organic shell of the AuNP and not its metallic core.

It has been previously reported that MMP-2, a gelatinase capable of degrading gelatin and its analogues, was detected in the cytoplasm of cancer cells in 75.6% cases (102 cases). Nakopoulou L, et al., “MMP-2 protein in invasive breast cancer and the impact of MMP-2/TIMP-2 phenotype on overall survival,” Breast Cancer Res Treat 2003, 77:145-155. Others have reported the enzymatic controlled release from PEGylated gelatin nanoparticles. Kim K J, Byun Y., “Controlled release of all-trans-retinoic acid from PEGylated gelatin nanoparticles by enzymatic degradation,” Biotechnol. Bioprocess Eng. 1999, 4:215-218.

Several works have been reported for coating AuNPs with appropriate polymeric agents for enabling cytoplasmic delivery. See, e.g., Hua et al., ACS Nano, 2010, 4 (9), pp 5505-5511, “Enhanced Gene Delivery and siRNA Silencing by Gold Nanoparticles Coated with Charge-Reversal Polyelectrolyte.” Using Cell TEM, reports prove naked AuNPs accumulation within endosomes or lysosomes upon internalization through phagocytosis. Reports on the cellular internalization of gelatin nanoparticles have primarily focused on confocal florescent microscopy to understand the destination of the particles within various cellular compartments. Studies have revealed that the majority of florescent labeled gelatin nanoparticles upon phagocytosis remain in endo-lysosomal compartments. In one study, florescence microscopy has shown PEG modified thiolated gelatin nanoparticles undergo endosomal escape for transfection of plasmid DNA. See, Kommareddy et al., Nanomedicine. 2007 March; 3(1): 32-42, “Poly(Ethylene Glycol)-Modified Thiolated Gelatin Nanoparticles for Glutathione-Responsive Intracellular DNA Delivery.”

Azimi et al. have reported gelatin nanoparticles as a delivery vehicle for bovine serum albumin Azimi et al., “Producing Gelatin Nanoparticles as Delivery System for Bovine Serum Albumin,” Iran Biomed J. 2014 January; 18(1): 34-40. The method formed 200-300 nm gelatin particles and studied limits of BSA loading. The article reported one step and two step desolvation methods to produce the gelatin nanoparticles. The BSA was reported to be released in a biphasic modulation characterized by an initial relatively rapid release period followed by a slower release phase. The release was attributed to water permeation through the hydrogel matrix and absorption by the GNP, GNP swelling, and diffusion of BSA molecules through the swollen GNP.

### SUMMARY OF THE INVENTION

A preferred embodiment provides a method for forming a multi-dimensional metallic nanoparticle and polymer/protein/gel carrier nanoparticle nanoconstruct. The number of metallic nanoparticles that attach to the carrier nanoparticle is controlled via microfluidics or via controlling the reactivity of the metallic nanoparticle (preferably by dampening its reactivity to thiols and limiting the number of available thiols on the carrier nanoparticle). In a preferred microfluidic method, a solution of thiolated polymer, gel or protein nanoparticles (which can include protein nanoparticles with existing available thiols, e.g. HSA) is introduced into a first microfluidic pathway. A solution of metallic nanoparticles is introduced into a second microfluidic pathway. The flows from the first microfluidic pathway and the second microfluidic pathway are combined to form the nanoconstruct. The flow rate and concentration are controlled to ensure that agglomeration is avoided and a predetermined limited number of metallic nanoparticles attaches to each carrier nanoparticle. In a preferred reactivity controlled method, the metallic nanoparticles are functionalized to dampen reactivity and a protein nanoparticle with limited available thiols is the carrier nanoparticle. Preferably, the metallic nanoparticles are one of gold, silver nanoparticles, palladium nanoparticles, platinum nanoparticles and iron nanoparticles. Preferably, the thiolated polymer or protein nanoparticles are gelatin nanoparticles

A preferred embodiment provides a multidimensional nanoconstruct. The nanoconstruct includes a three-dimensional protein, thiolated gelatin or polymer nanoparticle particle and exposed metallic nanoparticles bonded to outer surfaces of the particle. Preferably, the number of metallic nanoparticles is a predetermined number within a limited range that is a fraction of the predetermined number on either side (+/−) of the predetermined number.

### BRIEF DESCRIPTION OF THE DRAWINGS

FIG. 1 is a diagram of a preferred embodiment method for synthesizing a homogenous multidimensional nanoconstruct in controlled manner using fluidics;

FIG. 2 illustrates an experimental fluidics system employed to demonstrate a preferred method of synthesis according to FIG. 1;

FIG. 3 is a plot with data that illustrates the efficiency of integration determined by varying the concentration of Th-Gel NP (nanoparticle) and AuNP in the experimental formation of Gel-AuNP multi-dimensional nanoconstructs (MDN); TEM images confirmed FIG. 3 data showing that AuNPs at various concentrations were integrated to protein nanoparticles;

FIG. 4 is a graph with data that represents the efficiency of integration of AuNP normalized to per mg of nanoparticles as a function of concentration of AuNP per Mg of Th-Gel NP (mol/mg);

FIG. 5 is a plot illustrating MMP-2 assisted degradation of MDN (1 mg/ml) and release of ICG at 15 hrs incubation @ 37° C.;

FIG. 6 is a plot of data illustrating the charge reversal property of MDN post AuNP integration;

FIG. 7A is a plot of UV-Vis absorption spectroscopy of (a) DI Water, (b) AuNP, (c) shGel NP with encapsulated cy5-labelled insulin and (d) MDN with encapsulated cy5-labelled insulin; the UV-visible absorption spectrum confirms presence of two absorption bands;

FIG. 7B is a table of calculated theoretical number of gold nanoparticles that can be attached to the surface of a carrier gel nanoparticle;

FIG. 7C is a table including semi-empirical surface area available for accommodating the gel nanoparticles and AuNP per μSec;

FIG. 8 shows a preferred method for synthesis of HSA carrier/metallic nanoparticles and HSA-Au nanoparticles that is for synthesizing a homogenous multidimensional nanoconstruct in controlled manner using control of the reactivity of the metallic nanoparticles.

### DETAILED DESCRIPTION OF THE PREFERRED EMBODIMENTS

An embodiment of the invention is a multi-dimensional nanoconstruct of a polymer, gelatin or protein and a predetermined number within a limited range of metallic nanoparticles integrated with the polymer. The limited range is a fraction of the predetermined number on either side (+/−) of the predetermined number, as methods of fabrication of the invention can control the number of metallic particles per nanoconstruct. For preferred embodiment low (2-8 nanoparticles), medium (18-30 nanoparticles) and high (36-60 nanoparticles) predetermined numbers of particles respective ranges are the +/−=2 (if the number of nanoparticles is 2, then the +/− is 1), for medium +/−=5 and in case of high +/−=15. Polymer or gelatin nanoparticles in preferred embodiments form a multi-dimensional particle and the metallic particles are bonded to outer surfaces of the particle. Thiolated gelatin nanoparticles are used in preferred embodiments. Polymer nanoparticles, including PEGgylated gelatin that degrade to release the metal nanoparticles and or a payload encapsulated in the polymer nanoparticles can also be used. A plurality of the nanoconstructs is homogenous, meaning that each nanoconstruct in the plurality of nanoconstructs will have the same predetermined number within the limited range of metallic nanoparticles. In a preferred embodiment, the construct consists essentially of the polymer and the metallic nanoparticles. An embodiment of the invention is a method for fabricating a polymer and metallic nanoparticle constructs, that includes homogenous groups of nanoconstructs, with each nanoconstruct within the group having the predetermined number within a limited range of metallic nanoparticles integrated with the polymer. The limited range is established in a controlled and repeatable manner by preferred methods of fabrication of the invention.

To the best of our knowledge, no reports till the present invention as described in this application have shown gelatin nanoparticles within cellular compartments using transmission electron microscopy, which has been verified experimentally by the present inventors. A possible reason could be attributed to gelatin nanoparticles being prone to degradation within cells and thereby not easily detectable. Compared to metallic nanoparticles gelatin nanoparticles have relatively lower density for effecting contrast with cellular structures.

A preferred method of fabrication prepares metallic nanoparticles and polymer nanoparticles separately. Microfluidic mixing with precise control forms preferred nanoconstructs with the nanoparticles conjugated to polymer nanoparticles In preferred embodiments, metallic nanoparticles are mixed though nanofluidic delivery with thiolated gelatin nanoparticles that are delivered via a separate and independent microfluidic path. The gelatin nanoparticles can encapsulate drugs or other payloads and the metal nanoparticles can carry or be encapsulated with another payload. A nanoconstruct can therefore, for example, include multiple therapies, and the release of the therapies in vivo can be sequential. This provides a multi-drug delivery vehicle. Experiments used gold nanoparticles (AuNP). However, other metal nanoparticles are also “soft” in nature; in a similar fashion, meaning that thiol is a “soft” ligand. The present invention leverages thiols that enable interaction with metal nanoparticles, and controls the interaction to limit the number of nanoparticles that attach. In preferred embodiments, relative concentrations and interaction time of the carrier nanoparticles (larger gel, polymer or protein) with the metallic nanoparticle is controlled via microfluidic mixing. In other embodiments, the reactivity of the metallic nanoparticle and/or the carrier nanoparticle is dampened to limit the number of metallic nanoparticles that attach.

In preferred methods of fabrication, the fluidic delivery of the separate components precisely controls the number of metallic nanoparticles per nanoconstruct that is formed. In preferred methods, a preformed polymeric/protein nanocomponent is integrated to a preformed metallic nanocomponent in a manner that retains their respective unique properties, including any functionalities embedded in the polymeric nanocomponent or attached to the metallic nanoparticle component. The tunable surface functional properties of the metallic nanoparticle components provide a wide range of capabilities, while the polymeric/protein nanoparticle component provides high loading capacity. The simultaneous exploitation of the payload capacities of both components provides the opportunity to improve many applications such as sensors, diagnostics, probes and drug delivery systems. The ability to carry multiple “active ingredients” individually by the components provides the capability for development on drug delivery systems for complex diseases such as drug resistant cancers analogues.

A protein-metal or a polymer-metal nanocomplex of the invention provides the opportunity to widen the applicability of the individual components constituting the nanocomplex in areas of therapy, sensors, probes, catalysis and diagnosis upon simultaneous exposure of both classes of nanoparticles to surrounding environment. Physicochemical properties pertaining to individual components and combined derivative properties of the nanocomplex can be effectively exploited if the metallic component as well as polymer component can interact freely and individually with the external environment. This feature is distinct from a mere coating or encapsulation of the metallic component within a protein and polymer since encapsulation isolates the metallic nanocomponent from exposure to the external environment. Additionally, the physicochemical property of the polymer is not utilized when it serves as a mere encapsulate. In contrast, the integration of a preformed metallic nanoparticle to a preformed protein/polymeric nanoparticle provides preferred nanocomposites that allow the properties of both nanocomponents to be fully exploited.

Preferred methods provide controlled integration of metallic nanoparticles and protein nanoparticles via microfluidic mixing and leverage the affinity of reactive thiols on gelatin nanoparticles. Methods of the invention use microfluidic mixing to chemically conjugate gold nanoparticles onto the surface of gelatin nanoparticles in a controlled fashion. Gelatin nanoparticles are known for self-disintegration upon enzymatic action, which makes a produced nanoconstruct of the invention an excellent platform for delivery to the cytoplasm and/or nucleus of cancer cells. This feature has been demonstrated experimentally. Cellular studies indicated disassembly of surface conjugated gold nanoparticles from gelatin nanoparticles of a preferred experimental nanoconstruct upon internalization and prior to the self-destruction of the gelatin nanoparticle component after internalization at cellular level. The fabrication methods permit precise tuning of nanoconstructs that are formed, e.g. via integration of variously sized nanoparticles and materials. Preferred nanoconstructs can therefore be tailored as delivery systems with desired properties, and also tailored to have desired properties for in sensory applications, probe designs, catalysis and diagnostics.

The nanoconstructs of preferred embodiments can provide tuned surface properties. The number of metallic nanoparticles on the surface of the nanoconstruct can be selected within a narrow range, and capability of such precision can be leveraged to increase sensitivity for sensory and catalytic applications. The gelatin nanoparticles serve the function of a base particle, which will self-degrade through protease activity. The gelatin nanoparticles can carry a payload to enable exponential release of entrapped drugs with the addition of an enzyme. Within cells, a number of proteases play catalytic role for efficient conversion of protein for metabolic activity. For cytoplasmic delivery of AuNPs, the nanoparticle carrying AuNPs must first undergo endosomal escape. Thereupon, the primary requirement would be surface degradation of the gelatin nanoparticles inside the cytoplasm of the cells to initiate liberation of the surface conjugated AuNPs. MMP-2 along with other proteases present in the cytoplasm can trigger the release of AuNPs in addition to the release of the compounds entrapped within the particle. Preferred nanoconstructs of the invention can be delivered to cytoplasm of the cells.

Additional preferred embodiments provide albumin-derived nanoparticles (HSA-NP) with metal nanoparticles for drug delivery applications. A preferred method conjugates metallic nanoparticles within the albumin matrix as well as on the surface of HSA NP. The nanoconjugate has potential applications towards step-wise, multi-drug delivery. In preferred methods, HSA nanoparticles (HSA-NPs) were prepared and covalently modified with polyethylene glycol and encapsulated or surface conjugated with gold nanoparticles. The surface of AuNPS can be easily modified and more importantly these particles are non-immunogenic, biocompatible, and have shown substantial promise in biomedical applications and is a judicial choice for encapsulation purposes. Poly (ethylene glycol), or PEGs, are homo- or hetero-, mono- or bifunctional hydrophilic polymers were coated on the surface of gold nanoparticles to improve their circulation in blood, prolong drug half-life, and enhance pharmacokinetic properties and drug release

Preferred embodiments of the invention will now be discussed with respect to the drawings and with regard to experiments that were conducted. The drawings may include schematic representations, which will be understood by artisans in view of the general knowledge in the art and the description that follows. Features may be exaggerated in the drawings for emphasis, and features may not be to scale. The experimental examples will also illustrate broader features of the invention to those having skill in the art.

Experiments have demonstrated preferred nanoconstructs and preferred methods of fabrication. A set of experiments prepared gelatin and gold nanoparticles separately. The gelatin nanoparticles were thiolated. The gold nanoparticles were formed on the outside of a multi-dimensional nanoconstruct including the gelatin as a form of particle via microfluidic mixing. In experiments, separate syringes were used to precisely control the microfluidic delivery, and the number of gold nanoparticles, within a narrow range, per nanoconstruct. The experiments also tested surface conjugation of the nanoconstruct and conducted in vitro cell experiments to verify the potential for intracellular delivery to cytoplasm. Experiments also demonstrated that nanoconstructs can be formed with two separate predetermined sizes of nanoparticles. A second microfluidic integration was conducted with a second size of nanoparticles on nanoconstructs that had been formed with a first size of nanoparticles. The number of each of the two sizes of nanoparticles can be controlled within a narrow limited range.

While gold nanoparticles were used in most of the experiments, other metallic particles are used in other preferred embodiments. Other metallic nanoparticles that have affinity towards sulfhydryl group to form metal-SH bond are used as a component of other preferred nanoconstructs. Example includes but not limited to silver nanoparticles, palladium nanoparticles, platinum nanoparticles and iron nanoparticles. Silver nanoparticles were also demonstrated experimentally, as were complex MNDs with a plurality of sizes of metal nanoparticles.

Synthesis and Characterization of Gelatin Nanoparticles with Encapsulated Indocyanine Green.

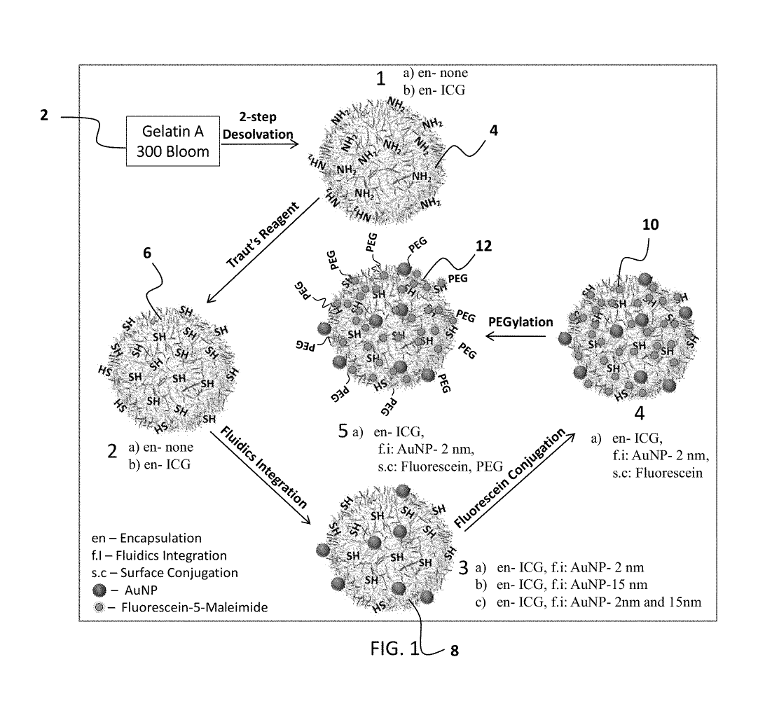

FIG. 1 illustrates a preferred synthesis method that was demonstrated experimentally. The method utilizes Gelatin A bloom 300 as a precursor to prepare Gelatin nanoparticles (1) using a two-step desolvation process. Specifically, Gelatin nanoparticles (Gel NPs) (1) prepared by 2-step desolvation method using Gelatin Type A (300 bloom) as a precursor to obtain 220 nm spherical nanoparticles. Primary amines of Gel NPs reacted with Traut's reagent (2-iminothiolane) to thiolate Gel NPs (2) at room temperature for 12 hours with pH of the solution adjusted to 8. Variable proportion of AuNPs were reacted with 2 by fluidics integration at a constant flow rate of 2.77 μl/sec in a mixing chamber followed by continuous stirring of the exited nanoparticles solution (3) at room temperature. Surface conjugation of 3 was performed by reacting fluorescein-5-maleimide at pH 7 with continuous stirring at 600 RPM for 2 hours at room temperature to obtain 4. PEGylation was performed using mPEG-Maleimide (Mw: 1000 Da) at 4° C. for overnight to obtain 5. Additional details of a particular experiment include: 500 mg of Gelatin A was first dissolved in 10 ml of de-ionized (DI) water at 50° C. After complete dissolution, 20 ml of acetone was added rapidly and the solution was stirred for 30 seconds. Higher molecular weight Gelatin precipitated in the first desolvation and the supernatant was discarded. The precipitate was dissolved in 10 ml DI water at 50° C. After complete dissolution, 8.5 mg of Indocyanine Green (ICG) was added and the pH was adjusted to 2.75 (±0.1) using HCL (1 N). In the second desolvation step, 20 ml of acetone was added drop-wise at a rate of 3 ml/min. The flow rate was maintained using dual infusion syringe pump (KD scientific, KDS 200). Transformation of the transparent green solution to a milky green solution indicated successful desolvation and formation of nanoparticles. After 10 minutes, 200 μl of 25% glutardehyde was added to the solution for crosslinking. The reaction was allowed overnight at 50° C. and the resulting nanoparticulate solution was centrifuged at 20,000 g for 30 minutes. The precipitate was then washed 5 times with DI water to remove excess ICG and glutaraldehyde. Sodium metabisulfite was added after first wash to quench unreacted glutardehyde and the reaction was allowed for 2 hrs. before the subsequent washings. The obtained nanoparticle solution was sonicated for 15 minutes and passed through 0.45 μm cellulose acetate filter and stored at 4° C. for further experiments. Fluorescein dye was used to mimic as a drug to show encapsulation ability of Gel NPs. Conjugation of fluorescein as evident from fluorescence microscopy and cellular studies shows that fluorescein is encapsulated with Gel NPs. Similarly, any drug can be encapsulated to Gel NP/HSA NPs. Fluorescein was conjugated to visualize cellular internalization of these nanoparticles, PEGylation of nanoparticles serves to aid biocompatibility, increases blood circulation time in vivo, prolongs drug half-life, and enhances pharmacokinetic properties and drug release. An example payload is biomolecule having efficacy (e.g. Chemotherapeutic molecules such as doxorubicin, cis-platin, gemcitabine, oxaliplatin are examples) for cancer treatment. Thiol containing drugs readily attach to the metallic nanoparticles.

In FIG. 1, fluid integration is leveraged as a way to control the reactivity between two different reactants. The carrier gel nanoparticles and the metallic gold nanoparticles are allowed to interact in defined proportion so that defined MDNs are obtained. Flow rate is used as a parameter to control the reactivity.

The gelatin, polymer or protein nanoparticles are larger carriers for the smaller metal nanoparticles. Typically, the core size of metallic nanoparticles is 2-10 nm and the carrier HSA NP is ˜180 nm. (Gel NP ˜215-220 nm). The gel nanoparticles are thiolated to provide for the “soft” attachment of the metal nanoparticles. Gelatin Type A used in the example experiments has a molecular weight range of 50000-100000 Daltons. The size of Gel NP is approx. 215-220 nm as analyzed by TEM and dynamic light scattering (DLS) analysis.

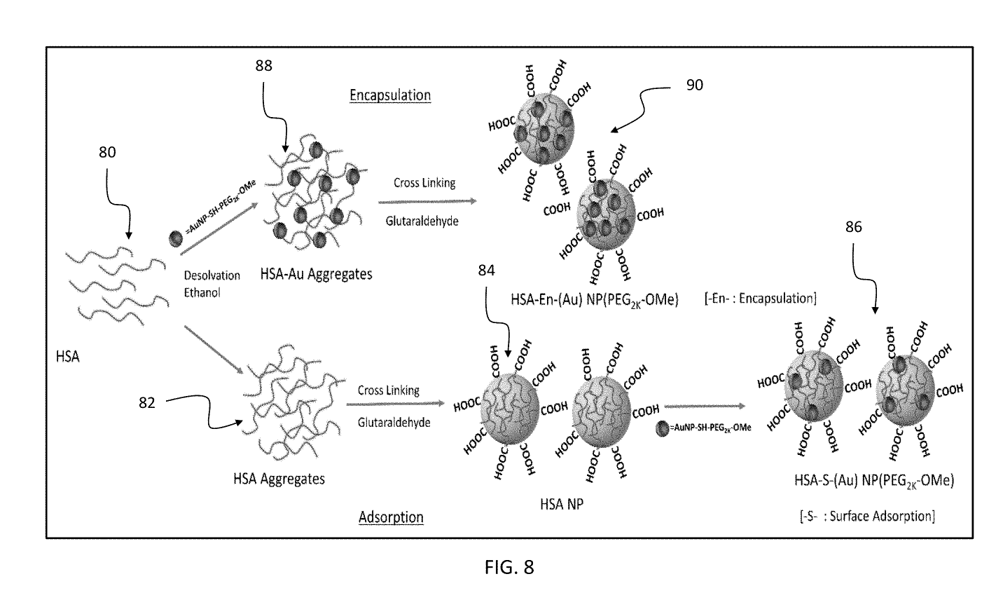

Protein nanoparticles can be used as carrier nanoparticles. Generally, proteins contain amino acid residues with free sulfhydryl groups. If such functional groups are not available, free amine functionality can be reacted with 2-iminothiolane (Traut's reagent) to obtain free thiols. As demonstrated by the FIG. 1 (and FIG. 8) experiments, the free thiols provide for the controlled attachment of metal nanoparticles.

Synthesis of Gold Nanoparticles

Two experimental methods were used to produce gold nanoparticles. Different reducing agents reduced gold salts to nanoparticles. Other alternatives for the gold nanoparticle production include methods disclosed, for example, in Katti et al., U.S. Pat. No. 8,333,994, Stabilized, biocompatible gold nanoparticles and method for making the same.

Borohydride Reduction:

Uniformly distributed gold nanoparticles of core size of ˜4-6 nm was synthesized using sodium borohydride as stabilizer. To synthesize the nanoparticles, 10 ml 0.13 M sodium borohydride was added dropwise to 1 ml of 0.1 M hydrogen tetra aurochlorate in 500 mL of DI water. Uniformity of the nanoparticles was achieved by controlled addition of the reducing agent. The core size of the particles was ˜5 nm.

Citrate Reduction:

Gold nanoparticles with 10 nm core size were synthesized by reducing the hydrogen tetra aurochlorate with sodium citrate. To 75 ml of boiling water, 1 ml of 10 mg/ml HAuCl₄ was added and allowed to heat further. When the temperature reached 110° C., 1 ml of 22 mg/ml of sodium citrate was added and the solution was stirred for 20 mins. The solution gradually developed the characteristic Wine red color and yielded ˜15 nm citrate stabilized gold nanoparticles. PEGylated gold nanoparticles-gold nanoparticles with core size of 2-3 nm prepared by THPC procedure, were pegylated using SH-PEG(2000)-OMe. To 45 ml of as prepared THPC stabilized gold nanoparticles were incubated with 4 ml of 7.5 mg/ml of SH-PEG(2000)-OMe. The reaction mixture was stirred overnight at room temperature. Excess of unreacted PEG was dialyzed using 70 kD membrane filter at the flow rate of 30 ml/min to obtain PEGylated gold nanoparticles.

Thiolation of Gelatin Nanoparticles and Determination of Degree of Thiolation.

Thiolation of the gelatin nanoparticles was performed by modifying the residual primary amine groups (NH₂) present in the cross-linked nanoparticles. In brief, 50 mg of Gelatin nanoparticles was incubated with 5 mg of 2-iminothiolane hydrochloride (Traut's Reagent) at room temperature for 12 hrs with constant stirring. The pH of the solution containing gelatin nanoparticles was adjusted to 8 prior to incubation. The primary amine groups converted to free reactive thiols providing gelatin nanoparticles 6 (FIG. 1) with free reactive thiols. Reactive thiols are necessary for covalent attachment of metal nanoparticles to the Gel NPs (as in the method of FIG. 1) or HSA NPs (as in the method of FIG. 8). The solution was repeatedly washed to remove excess Traut's reagent. Thiolated Gelatin Nanoparticles (SH-Gel NPs) were immediately used for subsequent integrations.

Multi-Dimensional Particle: Controlled Integration of AuNPs to SH-Gel NPs

Fluidics driven integration of the invention exposed reactive thiols present on the surface of the gelatin nanoparticles (SH-Gel NPs) to gold nanoparticles (AuNPs) in a controlled manner to produce the multidimensional nanoconstruct 8 (MDN). Two separate syringes containing solutions of 5 ml of SH-Gel NPs and 5 ml of AuNPs were mounted on a syringe pump. The composition of Sh-Gel NPs is varied from 0.25 mg/ml to 1 mg/ml and AuNP varied from 0.0125 mM to 0.1 mM. The syringes were connected to mixing chamber scavenged from Shimadzu HPLC (model 10AVP). A stainless steel connector pipe (100 μm ID) from the mixing chamber was used for connecting the outlet port. The collector vial was placed in a magnetic stir bar (300 RPM, room temperature). A constant flow rate of 2.77 μl/sec was maintained for both solutions. An intermediate investigation of the stock solution after 2.5 ml reaction was carried out using UV absorption spectroscopy (presence of AuNP or ICG containing SH-Gel NP) and size (for AuNP solution to determine the presence of SH-Gel NPs). This tested and ensured that neither of the particles diffused to the stock solutions. No significant changes relative to the stock solution were observed.

Surface Conjugation of Multi-Dimensional Particle to Fluorescein and PEGylation

The multi-dimensional particle containing solution was adjusted to pH 7.0 using sodium phosphate buffer. 10 μg of Fluorescein-5-maleimide was added to 0.5 mg/ml multi-dimensional particle solution to provide fluorescein conjugated MDNs 10. The solution was stirred at room temperature for 2 hours at 600 RPM. Thereafter, fluorescein conjugated multi-dimensional particle was subjected to 100 KDa filtration using to remove unreacted fluorescein. The obtained precipitate was re-suspended in pH 7 phosphate buffer solution and 100 μg of mPEG-Maleimide (Mw: 1000 Da) was added to produce PEGylated MDNs 12. The reaction was allowed overnight at 4° C. and the obtained suspension was washed and purified using DI Water.

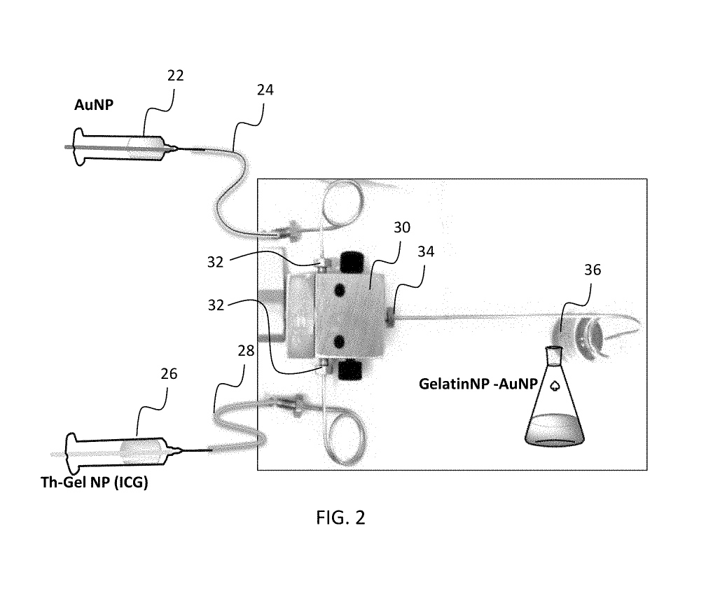

FIG. 2 illustrates a preferred microfluidics system used experimentally to perform the method of FIG. 1. A first source 22 (syringe pump) of AuNp nanoparticles injects a solution containing the metal nanoparticles into a first microfluidic path 24, in the form of a microfluidic tube. A second source 26 (syringe pump) of AuNp nanoparticles injects a solution containing the metal nanoparticles into a second microfluidic path 28, in the form of a microfluidic tube. The first and second sources 22 and 26 were flow controlled. The first and second sources 22 and 24 maintain selected predetermined flow rates into a microfluidic mixing chamber 30 via input ports 32. Reaction begins in the mixing chamber. Mixed solution exits an output port 34 of the mixing chamber 30 and is collected in a receptacle 36, which was a collector vial in experiments (represented both schematically and with an image in FIG. 2. The solution in the receptacle 36 is agitated, e.g., via stirring, to complete the reaction and formation of homogenous three-dimensional thiolated gelatin or polymer nanoparticle particle and exposed metallic nanoparticles bonded to outer surfaces of the particle.

Estimation of Encapsulation Efficiency.

Direct estimation of amount of ICG encapsulated within the nanoparticles was carried out using absorption spectroscopy. 1 ml of the synthesized nanoparticles was completely degraded using 2 mg/ml of protease. The degraded solution was centrifuged at 20,000 g for 30 minutes to ensure no precipitation of particles. The solution was then passed through 0.2 μm cellulose acetate filter and the filtrate was characterized for determining the ICG content using absorption spectroscopy at 780 nm. ICG standard curve was then used to determine the concentration of the analyzed filtrate. The encapsulation efficiency was determined using:

$$Encapsulation Efficiency ( \% ) = \frac{ICG concentration in filtrate \times total volume}{Initial ICG in the solution} \times 100$$

Three different experiments with an initial loading of 8.5 mg of ICG yielded an encapsulation efficiency of 42 (±2)%. The relative amount of ICG with respect to Gelatin NP content was estimated to be 34 μg of ICG per mg of Gel NPs.

The degree of thiolation was determined using Elman's test by measuring the absorbance of 3-thio-6-nitrobenzoate (TNB) at 412 nm. 300 μl of samples were taken and 30 μl of Elman's reagent was added to each sample. The solution was incubated for 15 minutes. Thereafter, in order to avoid the overestimation of free thiols, the reacted nanoparticles were centrifuged at 20,000 g for 30 mins and the corresponding supernatants were analyzed for TNB. The sulfhydryl presence was calculated based on the extinction coefficient provided in literature (14,150 M-1 cm-1). It was determined that the amount of free sulfhydryl groups saturates at approximately 60 μM at relative ratio of 0.1 (mg of traut's reagent per mg of gelatin nanoparticles).

Desorption Enthalpy

Indocyanine Green (ICG) is a water soluble compound to a limit of 2 mg/ml. Since the amount of ICG present in the nanoparticles subjected to release study is substantially lower than the solubility limit (<500 μg/ml), than either diffusion mechanism or related burst release must enable almost 99% release. However, this release pattern was not observed and the release saturation limit was observed well within 15 hrs. Estimating the desorption enthalpy for ICG-Gelatin NP (42 kJ/mol) further strengthens our claim that the resulting multi-dimensional particle would release substantial amount of encapsulated compound only when in-contact with a degradation agent such as proteases and enzymes. It is noteworthy to mention that the higher desorption enthalpy of a system, then the lower the release of the encapsulated compound. As mentioned in a previous report, a system with a desorption enthalpy of 45 kJ/mol released 1.4% at 1400 hrs while another system with 37 kJ/mol released 65% of the encapsulated compound within 100 hrs.

Multi-Sized Particle Integration

Experiments also demonstrated that a plurality of separate, predetermined sizes of particles can be integrated into a nanoconstruct of the invention. For this multi-sized particle integration, 0.001 mM AuNP (15 nm) was first integrated as described above. The multi-dimensional nanoconstruct/particle was collected. It included the 15 nm gold nanoparticles on its surface. This integrated nanoconstruct was re-loaded in substitution for the SH-Gel NPs. The second syringe consisted of a solution of 5 nm AuNP. The multi-dimensional particle was integrated sequentially to the smaller AuNPs through fluidics and the resulting solution was subjected to various characterizations including comparison with bulk reaction, transmission Electron Microscopy, Efficiency of Integration (EOI) and qualitative observations. The size and zeta potential of multi-dimensional particle and its analogues are given in Table 1

TABLE 1

| Com- | | | Com- | Size | Zeta |
|---|---|---|---|---|---|
| pound | Size | Zeta | pound | (PDI) | (SD) |
| 1 a) | 215 | 14.5(0.4) | 3 b) | 235(0.046) | 20.7(2.4) |
| 1 b) | 220 | 16(1) | 3 c) | 260(0.173) | 23.5(0.3) |
| 2 a) | 214(0.1) | 4.5(0.2) | 4 a) | 240(0.045) | 27.7(0.4) |
| 2 b) | 223 | 11.5 | 5 a) | 235(0.12) | 8 (0.4) |
| 3 a) | 228(0.1) | 18.8(0.5) | | | |

Table 1 includes a summary of physical properties of all synthesized compounds including the present MDN (multi-dimensional nanoconstruct). Results include Hydrodynamic Diameter (with poly dispersity index) and zeta potential (with standard deviation) of the synthesized compounds. The data reference hydrodynamic diameter and zeta potential of various compounds measured using Malvern Zeta Sizer Nano ZS. Charge reversal from positive charge of Gelatin nanoparticles to negative charge of Th-Gel NPs and MDN was observed. The size of the nanoparticles, however, did not undergo any drastic change considering the hydrodynamic size of Gel NPs now dominated over much smaller AuNPs integrated to its surface. Single peaks @˜220 nm and absence of smaller sized peaks indicated purified nanoparticles in the case of MDN.

In-Vitro Cell Experiments.

Cell experiments were conducted to test for cytoplasmic delivery in cancer cells. The experiments were conducted in vitro as a model in vivo behavior. The present constructs can be delivered to cancer via any of the following administrations: IV, IP or intratumoral. The natural drive for tumor uptake and retention is mediated by Enhanced Permeation& Retention. In addition, we have functionalized nanoconstructs with targeted molecules to target it to cancer cells

Cell Culture:

PC3 human prostate carcinoma cells (ATCC, USA) and A549 human adenocarcinoma epithelial cell (ATCC, USA) were grown in RPMI 1640 medium supplemented with 4.5 g/L D-glucose, 25 mM Hepes, 0.11 g/L sodium pyruvate, 1.5 g/L sodium bicarbonate, 2 mM L-glutamine, 10% heat-inactivated fetal bovine serum (Atlanta Biologicals, USA) and 0.1% v/v gentamycin. Cells were cultured in a humidified atmosphere of 95% air and 5% CO2 at 37° C. (Thermo Scientific, USA).

Dark-Field Fluorescence Microscopy:

A549 cells were seeded in 6-well plates (1×10⁶ cells/well). Cells were grown on a poly-L-lysine treated glass coverslip. 100 ul of each sample (normalized for o.5 mg/ml of SH-Gel NP) was incubated for 4 h at 37° C. in serum free media. After treatment, resulting cover slips were washed with PBS 1× to remove unbound particles and microscopic slides were prepared with DAPI nuclear stain. Slides were imaged using a polarized Dark-field fluorescence microscope at 20× and 40×.

TEM Cell Cross-Section Analysis.

PC3 cells were grown in 35 mm petri-plates (1×107 cells/well). 100 ul of (normalized for 1 mg/ml of SH-Gel NP) was incubated for 24 h and 6 h respectively, at 37° C. in serum free media. After treatment, cells were washed with PBS 1× eight times to remove unbound particles. TrypLE was used to remove cells and final cell pellet was stored in TEM fixative. 85 nm cross sections were made on a Carbon-Copper TEM grid using a Leica Ultracut Microtome and imaged using a JEOL JEM 1400 TEM at 80 KeV.

Results and Additional Data

Experiments demonstrate that multi-dimensional nanoconstructs according to an embodiment of the invention consisting of thiolated Gelatin nanoparticles (220 nm) encapsulated with Indocyanine Green (ICG) was integrated to AuNPs having core size of 5 nm using fluidics. The ICG was used to simulate a payload, such as a molecule to be delivered. A two step desolvation process was used for preparing Gelatin nanoparticles with 10% ICG loading during second desolvation step. Residual amines present on the surface of GelNPs after glutaraldehyde crosslinking were converted to reactive thiol using Traut's reagent to form thiolated gelatin NPs (shGelNPs). After purification and removal of surface bound ICG and thiolation of residual surface amine moieties, the nanoparticle solution was passed through a 0.45 μm cellulose acetate filter and used for the synthesis of multi-dimensional particle.

Gold nanoparticles with core size of 5 nm were synthesized using standard Sodium Borohydride reduction method. For integrating the two classes of materials, aqueous solution of AuNP was reacted with shGel NPs suspended in water. We observed that AuNP solution at a concentration of 0.2 mM when mixed drop-wise to shGel NPs (0.5 mg/ml) caused instantaneous aggregation and formation of interlinked ‘flower-type’ aggregates. Particularly, the importance of the present fluidic method was demonstrated for controlled integration for the synthesis of MDN. Without the present approach, a high concentration of AuNP (>0.2 mM) causes spontaneous aggregation due to AuNP-SH-Gel-SH-AuNP interaction during bulk reaction. A plausible hypothesis for such aggregation can be attributed to an interparticulate (AuNP-shGelNP-AuNP-shGelNP) linkage At lower concentration a of AuNP (<0.2 mM) aggregation does not occur. However, TEM images revealed highly non-uniform integration at the lower concentrations. In contrast, TEM images of the present MDN through controlled integration mediated via fluidics reveal homogeneous integration as well as non-aggregation.

Molecular dynamic simulation of a system having gold nanoparticles coated with thiol modified polymers (Thiol coated AuNPs) has simulated an instantaneous aggregation phenomenon, with the simulation showing the effect of alkane thiol coverage density, terminal group and water solvent on the aggregation of thiol coated gold nanoparticles. Computational details showed aggregation of 4 gold nanoparticles (thiol capped) within a time-scale of 100 ns. Meena Devi J., “Aggregation of thiol coated gold nanoparticles: A simulation study on the effect of polymer coverage density and solvent,” Comput. Mater. Sci. 2014, 86:174-179. While this is a mere polymer encapsulation of gold nanoparticles, the simulation results are consistent with the present nanoconstruct formation that can be postulated to result from interparticulate linkage forming large hybrid complexes. The invention is not bound to that theory, as the microfluidic formation produces the nanoconstructs.

As a control test, the AuNP solution was diluted to 0.1 mM and added drop-wise to 0.5 mg/ml of shGel NPs. At this point, no aggregation of any sort was visible. MDN formation was highly heterogeneous with majority of the gelatin particles remained without gold nanoparticles present on the surface Electron microcopy revealed non uniform heterogeneous integration. We believe that the shGel NPs which first comes in contact with the AuNPs in bulk mixture react and integrate spontaneously, while other shGel NPs remain unassociated with any nanoparticles. Gobbo et al (see background section) results with carbon nanotubes are not contradictory because of the different materials, dimensions and surface functionalities. This can explain their report of maleimide-gold nanoparticle conjugated on thiol functionalized single walled carbon nanotubes (swcnt) through bulk reaction. Gobbo et al also provided limited information on homogeneity and relative precursor content. The paper did not detail any concentration but was limited towards making gold nanoparticles surface functionalized with maleimide coated on carbon nanotubes fractionalized with thiol.

The present experiments demonstrate that the preferred fluidics-driven integration provides a higher degree of control over bulk reactions and provides controlled exposure of the reactants. AuNPs were integrated with shGel NPs with control over reactive precursor particles. AuNPs and shGel NPs in predetermined relative proportions were passed through 100 μm channels (Laminar flow, Rn=10 @ flow rate: 10 ml/hr) separately at individual flow rate of 10 ml/hr. While 40 ml/hr resulted in clogging of the channels, multi-dimensional particles obtained at 20 ml/hr were heterogeneous. Also, at 20 ml/hr, we observed diffusion of AuNPs within the GelNP channels leading to clogging and leakage after few minutes. However, at 10 ml/hr, controlled assembly of AuNPs to shGel NPs through convective self-integration was achieved in the connected mixing chamber. Larger channels will support larger flow rates. The collected multidimensional nanoparticles (multi-dimensional particle) were observed to be uniformly integrated.

The intermediate solutions was inspected to ensure that neither of the nanoparticulate solution diffused to their complementary stock solutions. There should be no or minimal cross diffusion of one solution from channel 1 into channel 2 (containing another solution). It is important that both nanoparticles within each solution enter outlet channel after interacting with each other and not diffuse into the other particle's inlet channel. The intermediate investigation of both stock solutions after 2.5 ml reaction was carried out using UV absorption spectroscopy and size (presence of AuNP or ICG containing shGel NP within complimentary channels). A sampling channel was created before mixing chamber. The solutions were removed and analyzed using UV/Vis spectrometry externally. No significant changes relative to the stock solution were observed. Thereafter, the unreacted AuNPs were removed through centrifugal process after overnight incubation at 4° C. UV-Vis spectra for the synthesized multi-dimensional particle revealed ICG (780 nm) and AU peaks (520 nm) indicating stable AuNPs even after removal of sodium borohydride surfactant. Oxidation of free thiol and highly reduced thiol reactivity ensured aggregation-free purification. The particles were then passed through 0.45 μm cellulose acetate filter.

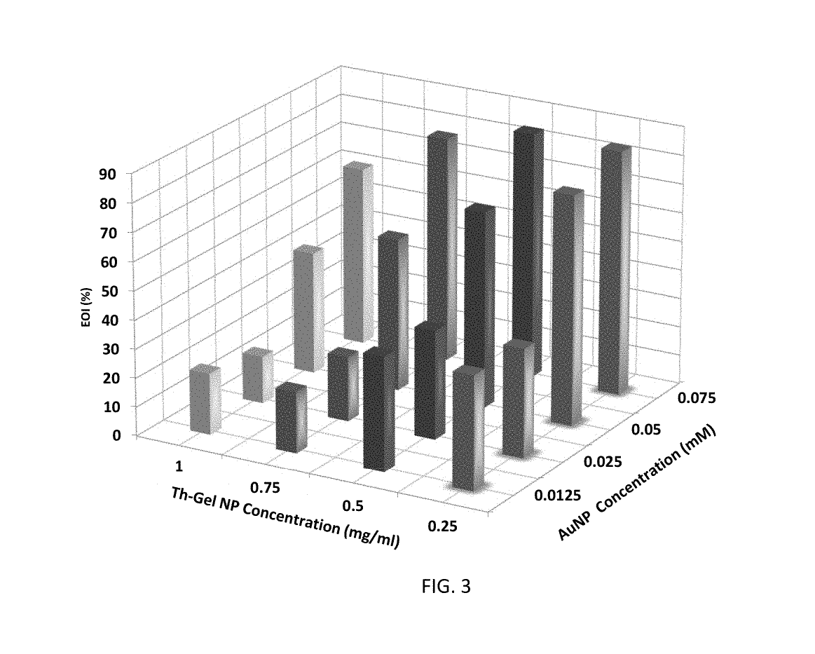

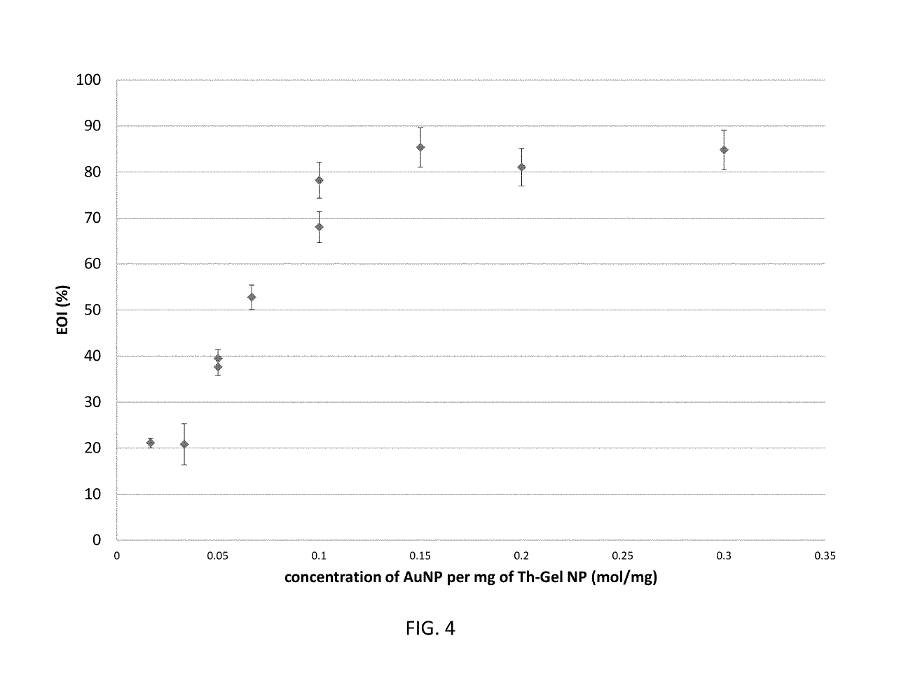

Control of the absolute amounts of AuNP for integration on the surface of multi-dimensional particle was also tested. Such defined nanocomplexes can play a significant role in advancing the platform for catalytic and sensory application of AuNP. Such control was demonstrated with increasing concentrations of AuNPs (0.0125 mM to 0.1 mM) and variable concentrations of shGel NPs (0.25 mg/ml to 1 mg/ml). The relative amounts of AuNP integrated to shGel NPs were determined by calculating the Efficiency of Integration (EOI). EOI was determined by analyzing the plasmon resonance of AuNP at 520 nm through UV-Vis absorption for each corresponding supernatants of various purified multi-dimensional particles. The relative proportionality derived from the UV-Vis absorption measurements indicated AuNP concentration dependent EOI FIG. 3. Increasing AuNP concentration yielded increased EOI (max EOI of 84% at 0.5 mg/ml Th-Gel NP and 0.075 mM SuNP). This was expected. However, a reversal of EOI with increasing amounts of shGel NPs is observed. Interestingly, we found a functional dependency of AuNP in the precursor solution per mg of shGel NP, defined as ratiometric value (Ŕ), on the resulting multi-dimensional particle (FIG. 4). The f(EOI) represented in the above figure is independent of each individual concentrations of AuNP integrated to Th-Gel NPs. A characteristic exponential profile of EOI with respect to mole of AuNP per mg of Th-Gel NP with asymptotic limit at approximately 85% was found. This suggests that there is, indeed a relationship for EOI and that it is a dependent function of AuNP concentration per Th-Gel NP concentration. The trend followed a sinusoidal curve for EOI with respect to AuNP conc./mg of Gel NPs with an initial lag below the Ŕ of 0.033. This is important considering the fact that the asymptotic limit of EOI is ˜85% @ Ŕ=0.15. The concentrations of the precursor nanoparticles used at this point were 0.075 mM and 0.5 mg/ml of AuNP and SH-Gel NP respectively. These results tend to demonstrate that increasing Ŕ would not yield any increase in EOI, explaining the EOI Reversal With Increasing Concentration of AuNP.

Transmission electron microscopy was conducted to corroborate the spectral value with images using the optimally fixed concentration of shGel NPs—0.5 mg/ml for various AuNP concentrations. TEM images revealed highly homogeneous integration of AuNPs to shGel NPs. Using image analysis (manual counting) the integration was relatively classified as high, medium and low wherein the high, medium and low comprises of 18-30 AuNPs, 9-15 AuNPs and 1-4 AuNPs integrated per shGel NPs respectively. Assuming equal distribution of the integrated AuNPs on the surface of the spherical shGel NPs, the absolute number of gold nanoparticles would therefore be twice the observed amount. The maximum number of gold nanoparticles per gelatin nanoparticle at Ŕ=0.15 was found to be 60 AuNPs per SH-Gel NP.

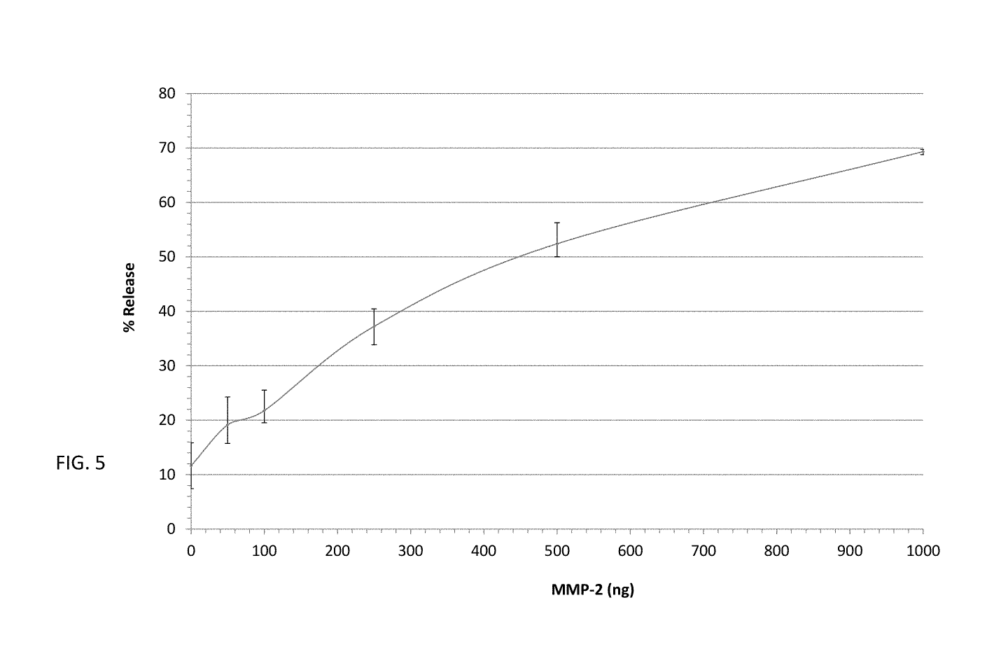

Characteristics of the nanoconstruct of the invention was also tested as a delivery system. Firstly, to understand the stability of the multi-dimensional particle considering gelatin nanoparticles degrades in presence of enzymes and proteases. Secondly, it was important that the encapsulated compound must release only upon an internal or external trigger. It has been previously reported that MMP-2, a gelatinase capable of degrading gelatin and its analogues, was detected in the cytoplasm of cancer cells in 75.6% cases (102 cases). Nakopoulou L, et al., “MMP-2 protein in invasive breast cancer and the impact of MMP-2/TIMP-2 phenotype on overall survival,” Breast Cancer Res Treat 2003, 77:145-155. The matrix degradation of the present nanoconstruct in presence of matrix metalloproteinases (MMP-2) was first confirmed by observing the release profile of ICG from multi-dimensional particle at 15 hr time-point incubated at 37° C. with various amounts of MMP-2. The experiments also demonstrated the release of ICG from the multi-dimensional particle with no presence of protease and cumulative results indicated an overall release of 10% in 48 hrs, an amount that is comparable to an earlier reported release from gelatin. Kim K J, Byun Y., “Controlled release of all-trans-retinoic acid from PEGylated gelatin nanoparticles by enzymatic degradation,” Biotechnol. Bioprocess Eng. 1999, 4:215-218. It was also found that the concentration of the enzyme is a prime factor that determines the overall degradability thus translating to the release of the encapsulated compound. The release profile of ICG from multi-dimensional particle was determined through absorption spectroscopy at 780 nm with increasing MMP-2 amount at pH 7.0. At MMP-2 amount of 1 μg, cumulative ICG release was 7 fold compared to no degrading enzymes (FIG. 5). In addition, semi-empirical calculations of release of ICG from the multi-dimensional particle demonstrated a high desorption enthalpy.

It is noteworthy to mention that—higher the desorption enthalpy of a system, the lower the release of the encapsulated compound. As mentioned in a previous report, a system with a desorption enthalpy of 45 kJ/mol released 1.4% at 1400 hrs while another system with 37 kJ/mol released 65% of the encapsulated compound within 100 hrs. Srikar R, et al., “Desorption-limited mechanism of release from polymer nanofibers,” Langmuir 2008, 24:965-974. Artisans will appreciate that the high desorption enthalpy shows that in the absence of any degradation agent, the encapsulated compound would remain intact within multi-dimensional particle. Assuming desorption of molecules from nanoparticles is comparable to that from the nanopores of nanofibers, the desorption enthalpy of Gel300A used in synthesizing multi-dimensional particle of interest was calculated using a previously reported desorption equation and clapeyron-like desorption law. The effective diffusion, Deff was calculated assuming the length, L=200 nm. Desorption enthalpy for multi-dimensional particle was estimated approximately 43 KJ. However, it is also important to note that this value is applicable only for molecules with properties similar to ICG and does not apply generally. Any new drug for delivery would have unique multi-dimensional particle-drug interaction which would alter the desorption enthalpy. Nevertheless, the value once established for a particular system would stand valid irrespective of the loading percentage or concentration of drug/gelatin. This further strengthens our claims that the chosen gelatin for multi-dimensional particle would degrade under active enzymes such as MMPs.

Cell tests established the versatility of the present multi-dimensional nanoconstruct/particle as a delivery system. The internalization was tested within prostate cancer 3 cell line (PC3) and lung adenocarcinoma cell line (A549)—to understand the mechanism and the destination of the various components of multi-dimensional particle upon internalization. Cytoplasmic delivery was confirmed. This result is important as several biomolecules as well as small molecules require cytoplasmic delivery. This feature is especially important for co-delivery of biomolecules such as siRNA along with its complementary drug. For cytoplasmic delivery, the particles preferably induce a so-called “proton-sponge effect” upon cellular internalization. Particles internalized via endocytosis should undergo endosomal escape and accumulate within cytoplasm by disrupting the endosomal membrane during late endosomal stage and avoid entry into lysosome.

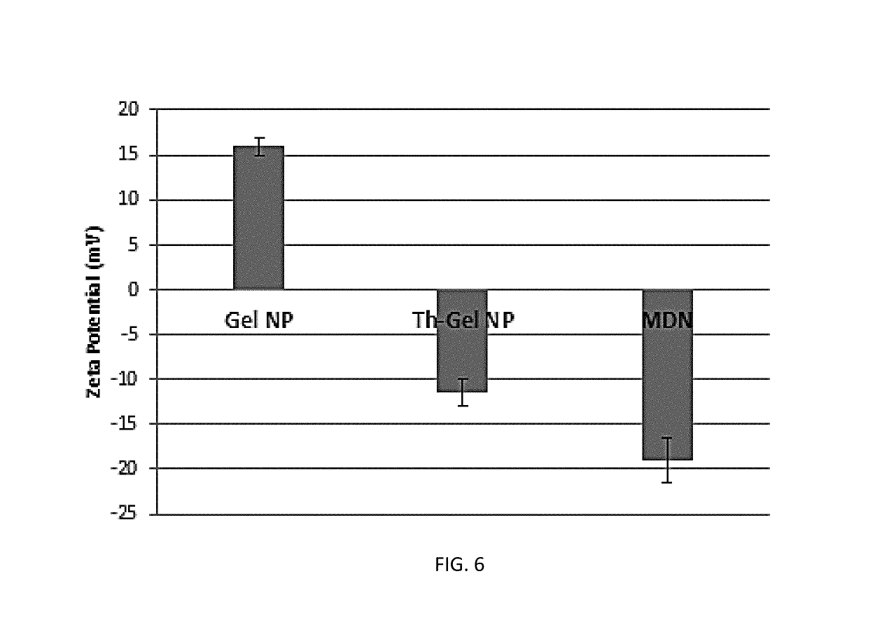

Components of the present multi-dimensional particle possess variable charge. Gelatin NP is positively charged but upon AuNP integration, the charge reverses to negative. This is demonstrated by the data in FIG. 6. The positively charged Gel NPs undergo charge reversal to negatively charged Th-Gel NPs upon thiolation and experience increased negative zeta potential upon integration with AuNPs to form MDN. One factor responsible for effecting the proton sponge effect is known to charge reversal of the particles. Change in the surface charge of particles from negative (pH 7) to positive (pH 5) can enable endosomal escape. Naked AuNPs do not exhibit charge reversal properties. This property affects induction of ‘proton sponge effect’, leading the AuNPs to localize within endosome or lysosome after internalization.

The present multi-dimensional nanoconstruct possesses the charge reversal property necessary for the desired localization. The ζ of multi-dimensional particle changed from −15 mV at pH 7 to +14 mV at pH4. This charge reversal can induce the proton sponge effect and enable endosomal escape. For determining the fate of multi-dimensional particle and its uniqueness compared to standalone AuNPs, we incubated Prostate Cancer cell line PC3 (24 hrs incubation) and A549 (24 hrs) with multi-dimensional particle. In case of PC3 cells, we observed an interesting phenomenon. After internalization of multi-dimensional particle and endosomal escape, we observed AuNPs agglomerated together within cytoplasm as well as near the endoplasmic reticulum of the cells. The particles starts degrading from outermost surface liberating its surface component i.e. AuNP However, in one particular section, we observed what could be the pattern of intra cellular trafficking of multi-dimensional particle occurring in 3 stages. In stage 1, after endosomal escape of multi-dimensional particle, the gelatin particles undergo superficial surface degradation within cytoplasm. The surface of the nanoparticles is the first phase contact with the external environment of the cells. The AuNPs present on the surface gets liberated from the gelatin nanoparticles. In stage 2, this liberated AuNPs cluster together within cytoplasm. Specifically, the gelatin particles undergo complete surface degradation and partial overall degradation. The liberated AuNPs cluster then together. Gelatin nanoparticles continue to degrade completely in stage 3 enabling localization of AuNPs within cytoplasm or transmigrate near endoplasmic reticulum. Clustered AuNPs localized near endoplasmic reticulum (ER) were observed at several locations within cells. Without being bound by the theory or limiting the invention, we believe that the encapsulated compounds are released within the cells considering “near-complete” degradation of gelatin particles. If confirmed, then the release of the compound is degradation-assisted occurring via enzymatic cleavage as proven earlier using in-vitro release studies with MMP-2 protein. The observed components of the multi-dimensional particle would then be located in the cytoplasm, a location that promotes endosomal escape of AuNPs. Minimal particles were observed entrapped within lysosome or endosome. In the case of multi-dimensional particle incubated within A549 cells, we observed AuNP component of multi-dimensional particle after stage 2 were transported within the nucleus of the cells, clustered at several locations within the nucleus. Some AuNPs were also observed in the cytoplasmic region. However, since we did not observe relatively high amount of gelatin NPs within A549, we decided to observe the cells incubated with multi-dimensional particle via fluorescence microcopy.

To observe the co-localization of multi-dimensional particle, fluorescein 5 maleimide (Fl5M) was conjugated to residual reactive thiol of multi-dimensional particle after AuNP integration to form multi-dimensional particle-Fl5M. Co-internalization of Gelatin nanoparticles and AuNPs was determined by incubating Non-small cell lung cancer (A549) cells with multi-dimensional particle-Fl5M for 4 hrs along with appropriate controls. Dark field and fluorescent microscopy revealed clusters of AuNPs and Fluorescent moiety internalized within cells. Fluorescein conjugated on the multi-dimensional particle, however was observed in majority of the cells. The results from fluorescence imaging suggested that the gelatin nanoparticles underwent degradation and hence its presence could not be completely detected or profiled using TEM imaging. Moreover, fluorescein quantification from within cellular boundaries (250 cells) using image analysis (Biotek cellular imaging software) revealed 142% increase in fluorescein signal emanating from multi-dimensional particle-FLSM compared to free fluorescein. shGel NP that is conjugated with Fl5M and Pegylated multi-dimensional particle-Fl5M showed 84% and 54% increase respectively. The quantification proves that multi-dimensional particle internalizes more compared to free shGel NPs. It is also known that PEGylated compounds' internalization in-vitro is generally lower than their nascent counterparts. Furthermore, considering high stability of multi-dimensional particle and negligible ‘free’ AuNPs, it is fair to conclude that internalization of multi-dimensional particles within the cell occurred as one stable compound.

It is noted that the destination of the multi-dimensional particles is dependent on the morphology and properties of the cells. The rate of degradation and proximity of multi-dimensional particles to nucleus post internalization can affect the destination. Without being bound by the theory, we believe that if the external boundary of cytoplasm is relatively closer to the nucleus, the time taken for the particles to degrade would decide the fate of the particles, i.e. if the particles end up in the cytoplasm or inside the nucleus due to the positively charged gelatin matrix. A detailed understanding of the targeted cells can aid the design of particular multi-dimensional nanoconstructs of the invention, which is provided by the microfluidic fabrication methods of the invention. A particular degradation profile can be achieved with control of the concentration of the gel nanoparticle based on the targeted cell. Even in cases where cytoplasmic delivery is not achieved, the multi-dimensional particles of the invention provide unique characteristics that are not provided by the separate gel or gold nanoparticles.

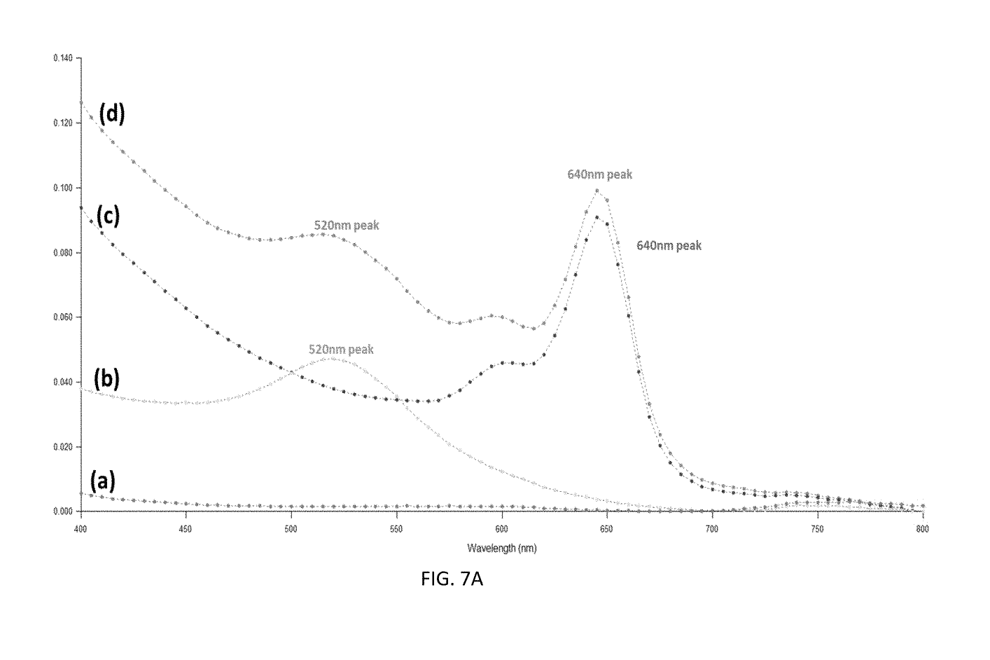

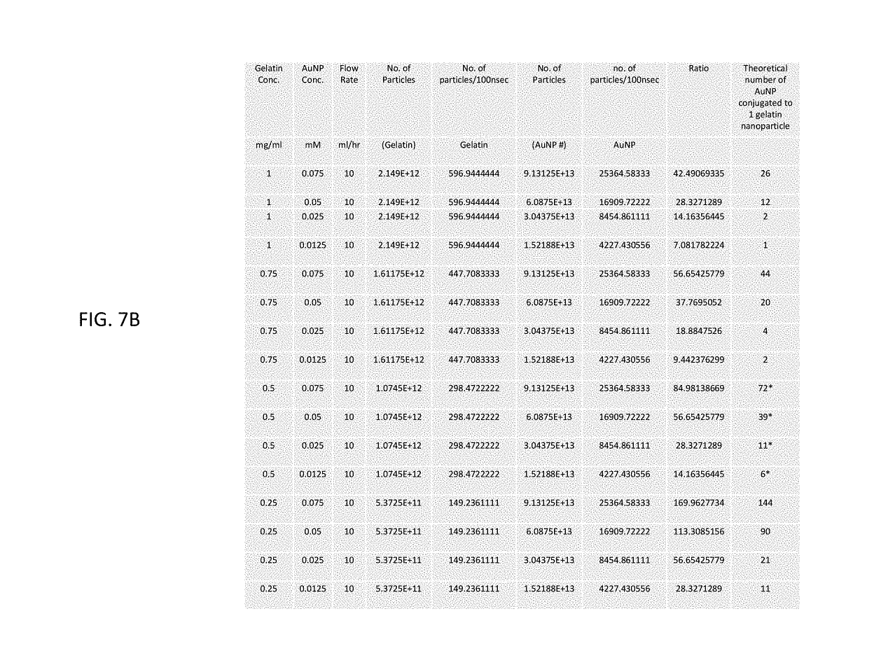

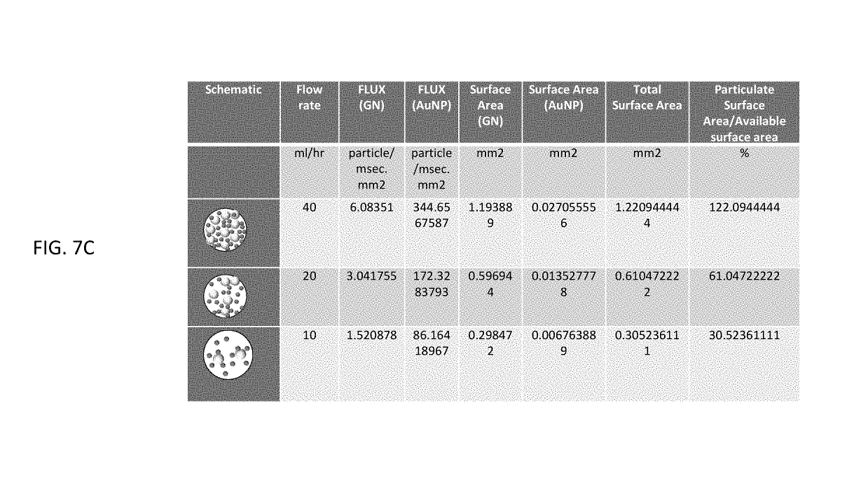

The Gelatin-gold nanoparticulate system is just an example for multi-dimensional particle platforms of the invention. Other metallic nanoparticles (M) which have affinity towards sulfhydryl group to form M-SH bond can be used. Silver nanoparticles, palladium nanoparticles, platinum nanoparticles and iron nanoparticles are used in other embodiments. In addition, the microfluidic formation methods permits more than one type and/or more than one size of metallic nanoparticle to be integrated in the nanoconstruct by sequential addition of nanoparticles as described above. An example more complex system containing multi sized metallic nanoparticles or another metallic nanoparticle on a given nanoparticle (Gelatin NP in this case) can also be designed and was demonstrated experimentally. Encapsulation of small molecule ICG has been used in the experiments. However, larger molecules like insulin can also be encapsulated. As a encapsulated cy5 labelled insulin within multi-dimensional particle and proved its presence within multi-dimensional particle using Optical imaging and UV-Vis absorption spectroscopy, which data is shown in FIG. 7A. AuNP and cy5-insulin shows characteristic peak at 520 nm and 640 nm respectively. MDN comprising both AuNP and cy5-insulin shows both 520 nm and 640 nm peaks.

Additional details and conclusions regarding microfluidic control.

Experiments according to FIG. 1 demonstrated fluidics mediated integration of metallic nanoparticles onto a larger carrier nanoparticle. The controlled microfluidic interaction of nanoparticles yields a compositional uniform multidimensional nanoparticle. In the example that demonstrated the integration of gold nanoparticles on gelatin nanoparticles, the composition of the nanocomposite is controlled by reacting predetermined number of gold nanoparticles to a known number of thiolated gelatin nanoparticles at any given time within a defined cross-sectional area. The microfluidics process demonstrated control such that nanocomposites of different composition were fabricated: [gelatin nanoparticles−(gold nanoparticles)x] where x average=2, 12, or 25. The nanocomposites were further surface conjugated with organic molecules such as fluorescent dye or polyethylene glycol (PEG) molecules.

The experiments demonstrated covalently integrated gold nanoparticles (AuNPs) on gelatin nanoparticles (GN) to form a library of 3DNC with general formula: [GN-(AuNP)x] x=1-4, 9-15, or 19-30 (FIG. 2). In the [GN-(AuNP)x], AuNPs are bonded through thiols on to the surface of GN. The biological property of [GN-(AuNP)x] synthesized in the experiments resembles that of molecules fabricated using the biomolecule-mediated self-assembly process. However, the present micro-fluidics route provides extraordinary control in developing nanomaterials with uniform composition and unique spatial arrangement. To covalently attach AuNPs to GN, it is necessary to modify the surface of GN with ligands to impart selective reactivity toward AuNP for forming covalent bond. Importantly, the post surface modification of GN should retain the stability and solubility to enable unfettered interaction with AuNP. The experiments converted the amino groups on the surface of GN, using Traut's reagent, to thiol groups. This functional group conversion increases the reactivity of GN toward AuNP, while retaining the integrity, stability, and solubility of GN. Without microfluidic control, however, the high reactivity of thiol modified GN toward AuNP led to massive nondirectional aggregation with precipitation. We determined that controlling the interaction kinetics between particles to less than 100 ns scale could produce a controlled integration, which was achieved with the microfluidic approach. The experimental microfluidics system included channels of cross-sectional diameter of 100 μm, and the length was fixed as 10 cm from the mixing chamber. As these dimensions are fixed, the flow rate of the reactants could be varied so that at the given time (100 ns or less) the number of nanoparticles interacting with each other in mixing chamber is controlled. For example, the theoretical number of AuNPs interacting per GN-SH at a time scale of 100 ns was calculated to be 72. FIG. 7B is a table that shows the calculated numbers.

To control the number of AuNPs interacting with GN, the flow rates of constituents in the fluidics system are varied to 10, 20, and 40 mL/h, and the resultant products were analyzed. On the basis of this flow rate at fixed concentration of the reactants, we theoretically calculated the number of NPs flowing through the mixing chamber in μm² cross-sectional area per μs and are presented in the following table.

| | | | Area of NPs |
|---|---|---|---|
| | AuNP | GN | with respect to |
| Flow Rate | (particle/ | (particle/ | area in mixing |
| (mL/h) | μs-μm²) | μs-μm²) | chamber (%) |
| 10 | 86 | 1.52 | 32 |
| 20 | 172 | 3.04 | 65 |
| 40 | 344 | 6.08 | 125 |

Even though the mixing chamber is three-dimensional, the comparison could be better attained with two-dimensional parameters. For example, at the flow rate 40 mL/h of reactants (6.08 GN/μs·μm2 and 344 AuNP/μs·μm2), the overall surface area of the particles occupying the space in the channels 1.25 μm² exceeds the available surface area of the channel leading to clogging of the fluidic channel. FIG. 7C shows data regarding the semi-empirical limit. Flux of the particles was calculated using the known parameters of velocity, area and the number of particles. The derived value of the flux was used to estimate available area within the mixing chamber with simplistic 2D derivation. The 2D schematic is a representation of the available area for the nanoparticles to undergo integration. At 40 ml/hr and 20 ml/hr, the flow rate exceeds the flux of particles (given the constant available surface area) and leads to agglomeration over time (normally within seconds). At 40 mL/h, the flow resulted in nontractable aggregated material. As a next step, we reduced our flow rate to 20 mL/h which led to controlled interaction with improved particle size; however, the clogging could not be controlled with time (particles occupying ˜65% of the available surface area). Finally, at 10 mL/h flow rate, controlled assembly of AuNPs to GN through convective self-integration was achieved in the connected mixing chamber. The collected 3D nanocomposite was observed to be uniformly integrated. These results confirm that by controlling the rate of interaction of nanoparticles using the micro fluidics-system controlled nanocomposites can be formed by control of the concentration, surface properties and interaction time/flow rates. With experimental data, we estimated the range of concentration ratios [GN]:[Au], at which the fluidics system would yield a well-defined 3D [GN-(AuNP)ₓ], comprising of x=1-4, 9-15, and 19-30. Assuming equal distribution of the integrated AuNPs on the surface of the spherical GN-SH, the absolute number of gold nanoparticles would therefore be twice the observed. The maximum number of gold nanoparticles per gelatin nanoparticle at Ŕ=0.15 would be 60 AuNPs per GN-SH. Theoretical number of AuNPs that can be bound to GNs was very close in correlation to TEM observations, as show in the table of FIG. 7B.

We also investigated the ability of the carrier nanoparticles to encapsulate molecules within gelatin matrix and also their ability to release it with external trigger such as enzymes. For this study, we used indocyanine green (ICG) as a model encapsulant owing to their absorption at 780 nm for easy and accurate analysis. We encapsulated ICG (20 mg/g of GN) within the gelatin. Encapsulation of ICG within gelatin nanoparticles was performed as per previous reports.20 Briefly, the amino groups on the surface of GN(ICG) were converted to thiol and covalently conjugated with AuNPs using fluidics process to yield 3DNC(ICG). Encapsulated ICG was retained with no release through out the different chemical modifications. The encapsulation and loading content of our system were determined to be ˜40% and ˜5% (w/w). studied the release characteristics of ICG from 3DNC(ICG) under different biological media; the results show that ICG is intact within the nanoparticle and only less than 10% released during 48 h of study period conforming to previous studies that have also indicated that the diffusion mediated release from gelatin saturates at ˜15%. We further studied the release characteristics of ICG from under different biological media in the present nanoconstruct; the results show that ICG is intact within the nanoparticle and only less than 10% released during 48 h of study period conforming to previous studies that have also indicated that the diffusion mediated release from gelatin saturates at ˜15%. We also determined that the gelatin carrier nanoparticles will degrade under active enzymes such as MMPs. Our studies further indicate that AuNPs present on the surface of 3DNC still provide the stereospecific access for MMP-2 to disintegrate gelatin.

FIG. 8 shows a preferred method for synthesis of HSA nanoparticles and HSA-Au nanoparticles. The FIG. 8 method controls the number of metal nanoparticles that attach to a surface of a protein nanoparticle (HSA) by controlling the reactivity of the metal nanoparticles. The FIG. 1 method controlled the number of nanoparticles by controlling the concentration and duration of interaction with microfluidics. Each of the methods keeps the number of metal nanoparticles below a number that will cause conglomeration. The FIG. 8 method utilizes the reactivity of fully functionalized gold nanoparticles and natural thiol present in HSA. For example, native gold nanoparticles are highly reactive and mixing yields a polymer. AuNP functionalized with molecules on the surface result in similar polymers. However, AuNPs fully functionalized on the surface dampens the reactivity appropriate to react with one or two thiol molecules. Thus, AuNPs fully functionalized (with possibility to further add one or two surface conjugations) enable conjugation of thiols present in HSA. HSA-nanoparticles contain only few thiol molecules on the surface, which are sufficient to react with the functionalized AuNP that have dampened reactivity. The FIG. 8 method includes 1) In Situ encapsulation of AuNPs to dampen reactivity and 2) Surface Adsorption of AuNPs on HSA NPs. Both methods produce multi-dimensional nanoconstructs of the invention without the spontaneous aggregation of uncontrolled reaction between the metallic nanoparticles and the carrier nanoparticle. Unlike gelatin, HSA has only 35 free sulfhydryl groups that may participate in forming gold-thiol interactions. Experiments also confirmed that the present nanoconstructs internalize effectively within cells, which was confirmed with TEM images.

In FIG. 8, Human Serum Albumin (HSA) (1) solution at pH 8-8.5 filtered vis 0.22 μm cellulose acetate filter subjected to desolvation using ethanol at flow rate of 1 ml/min at room temperature with continuous stirring at 500 rpm. During desolvation, AuNP-PEG solution added for in situ encapsulation of AuNPs to form HSA-Au aggregates (2). These were further crosslinked for 24 hours at room temperature using 8% aqueous glutaraldehyde to obtain 3. In surface adsorption method, HSA NPs (5) prepared similarly by desolvation process and crosslinked using gluteraldehyde. Surface adsorption to 5 was performed using AuNP-PEG_{2K} at room temperature under continuous stirring at 750 RPM for overnight to obtain HSA-AuNPs (6).

Gold nanoparticles were encapsulated in situ (HSA-En-(Au) NP(PEG_{2K}-OMe)) as well as physio adsorption technique (HSA-S-(Au) NP(PEG_{2K}-OMe)). The constructs in accordance with FIG. 8 provide targeted protein-metal nanoconjugates for multi-drug delivery. Specifically, the multi-drug delivery nanoconstructs contain two components—one protein based and the other metal based. Protein based NPs encapsulate a first drug. Metal nanoparticles covalently attach a second drug to the surface of the nanoparticles. The first and second drugs can be different drugs, which permits delivery of two different drugs. The first and second drugs can be the same drug, to achieve a two-stage release of the same drug. Whether the first and second drugs are the same or different, the multi-drug delivery nanoconstructs form a delivery vehicle to release the drug in different stages within cellular structures.

Synthesis of HSA Nanoparticles.

As represented in FIG. 8, Human Serum Albumin (HSA) solution 80 was prepared at the concentration of 100 mg/mL in 10 mM sodium chloride solution. Approximately 250-275 ul of 0.1M NaOH was added to adjust the pH to 8-8.5. The solution was filtered using 0.22 um Cellulose Acetate Filter to produce a solution with HSA aggregates 82. To this solution 4 mL ethanol was added at 25° C. at the flow rate of 1 ml/min under continuous stirring at 500 rpm. This process completes protein desolvation and the formed nanoparticles were stabilized by crosslinking using 56 μL of 8% aqueous glutaraldehyde solution. An HSA nanoparticle solution 84 stirred at 500 rpm for 24 hour and purified HAS nanoparticles were obtained by centrifugation at 25° C. with a speed of 16,100 g for 20 min for two times and redispersion in 1 mL 1×PBS using vortexing and ultrasonic water bath for 5 min.

Synthesis of Gold Nanoparticle.

Gold nanoparticles were prepared by reduction of Gold (III) Chloride trihydrate (HAuCl_{4.3}H₂O), using tetrakis (hydroxymethyl) phosphonium chloride (THPC), as the reducing agent. To 45 mL of DI water, 0.5 mL of freshly prepared 1 M NaOH and 1 mL of THPC solution were added. THPC solution was prepared by adding 12 tit of 80% THPC in water to 1 mL of DI water. This solution was continuously stirred for 5 min at room temperature. Gold (III) Chloride trihydrate (HAuCl_{4.3}H₂O) 2 mL of 25 mM was added rapidly to the stirring solution. The color of the solution changed from yellow to colorless to dark brown, indicating the formation of THPC stabilized gold nanoparticles. The reaction was allowed to stir for 15 min at room temperature.

PEGylation of THPC stabilized Gold Nanoparticles: To the solution of THPC stabilized AuNPs were incubated with 2 ml (15 mg/ml) of carboxymethyl-PEG-thiol (Mol. Wt. 2000). The reaction mixture was gently stirred overnight at room temperature. To remove unreacted PEG and reactants, the mixture was then dialyzed against distilled water for 24 hrs using a cellulose membrane with a cut-off size of 70 kDa for three times.

Surface Adsorption of AuNP on HSA nanoparticles. The AuNPs were attached to the surface of the HSA nanoparticles to form a HSA-AuNP nanoconstruct (6) FIG. 8. This was accomplished by simple mixing of AuNPs to HSA NPs. Human serum albumin (HAS) inherently contains free sulfhydryl groups that forms covalent interaction with AuNPs. Unlike with the gel nanoparticles, there is therefore no need to thiolate the HSA nanoparticles. In the method of FIG. 1, in case of Gel NPs, the amino groups were thiolated using 2-iminothiolane (Traut's reagent) and then conjugated to AuNPs. The method of FIG. 8 does not require the control of the mixing as in FIG. 1, and can use simple mixing. The control is achieved in FIG. 8 via damping the reactivity of the metallic nanoparticles and providing a carrier nanoparticle with thiol reactivity such that the combination limits the number of smaller metallic nanoparticles that attach to the larger carrier nanoparticles. Compositions on the surface of the metallic nanoparticles and carrier nanoparticles are selected to limit the number of metallic nanoparticles that attach to each carrier nanoparticle. After HSA NPs were prepared by protein desolvation and crosslinked using glutaraldehyde, these nanoparticles (20 mg/ml) were incubated with THPC stabilized PEGylated AuNPs (10 mg/ml) for overnight at room temperature under continuous stirring for 750 rpm. The nanoparticles were purified by centrifugation at 25° C. with a speed of 16,100 g for 20 min for two times and redispersion in 1 mL DI water using vortexing and ultrasonic water bath for 5 min

FIG. 8 also shows encapsulation of AuNP-PEG in HSA NPs. Human Serum Albumin (HSA) solution was prepared at the concentration of 100 mg/mL in 10 mM sodium chloride solution to provide the HSA solution 80. Approximately 250-275 ul of 0.1M NaOH was added to adjust the pH to 8-8.5. The solution was filtered using 0.22 um Cellulose Acetate Filter. This solution was incubated with AuNP-PEG (2.5 mg/ml) and incubated at room temperature with continuous stirring at 500 rpm for 3.5 hrs to provide a HAS-Au Aggregate solution 88. To this solution 4 mL ethanol was added at 25° C. at the flow rate of 1 ml/min under continuous stirring at 500 rpm. This process completes protein desolvation and the formed nanoparticles were stabilized by crosslinking using 56 μL of 8% aqueous glutaraldehyde solution to provide encapsulated nanoparticle solution 90. Nanoparticle solution was allowed to stir at 500 rpm for 24 hour and were purified by centrifugation at 25° C. with a speed of 16,100 g for 20 min and redispersion in 1 mL 1×PBS using vortexing and ultrasonic water bath for 5 min.

The experiments of FIG. 8 demonstrated the ability to control the reactivity between larger carrier nanoparticles and metal nanoparticles to achieve the same effect of microfluidics as in the FIG. 1 experiment to limit the number of metallic nanoparticles that attach to each carrier nanoparticle to a number below the number that will produces spontaneous aggregation. Simple mixing has conventionally resulted in agglomerates and polymeric components. Fluidic integration as shown via the FIG. 1 experiments avoids agglomerates and polymeric components via microfluidic control. Similarly, the FIG. 8 experiments control reactivity of fully functionalized gold nanoparticles and natural thiol present in a protein carrier nanoparticle (HAS). Native gold nanoparticles are highly reactive and mixing yields a polymer. AuNP functionalized with molecules on the surface result in similar polymers. However, AuNPs fully functionalized on the surface dampens the reactivity appropriate to react with one or two thiol molecules. Thus, AuNPs fully functionalized (with possibility to further add one or two surface conjugations) enable conjugation of thiols present in HSA. HSA-nanoparticles contain only few thiol molecules on the surface, which thereby controls the number of metallic nanoparticles that attach.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method of forming a multi-dimensional metallic and carrier nanoconstruct, the method comprising interacting a solution of metallic nanoparticles with a solution of gelatin, polymer or protein carrier nanoparticles, wherein the carrier nanoparticles comprise a free thiol, and the method further comprises controlling the number of metallic nanoparticles that attach to each carrier nanoparticle via microfluidic control or reactivity control.

2. The method of claim 1, wherein the control is microfluidic control, and the method comprises:
introducing a first solution of the carrier nanoparticles into a first microfluidic pathway;
introducing a second solution of metallic nanoparticles into a second microfluidic pathway;
mixing flows from the first microfluidic pathway and the second microfluidic pathway to form the nanoconstruct while controlling the flow rates and concentrations of the first and second solutions to limit interaction time to a level below which agglomeration occurs.

3. The method of claim 1, wherein the metallic nanoparticles comprises one of gold nanoparticles, silver nanoparticles, palladium nanoparticles, platinum nanoparticles and iron nanoparticles.

4. The method of claim 1, wherein the carrier nanoparticles comprise comprise thiolated gelatin nanoparticles.

5. The method of claim 1, wherein the metallic nanoparticles comprise nanoparticles having an affinity towards sulfhydryl group to form a metal-SH bond.

6. The method of claim 1, wherein said interacting comprises introducing the first and second solutions into a mixing chamber at a predetermined flow rate and concentration.

7. The method of claim 6, wherein said flow rate and concentration is selected to achieve a predetermined number of particles per carrier nanoparticle.

8. The method of claim 6, wherein said metallic nanoparticles comprise a first predetermined size of nanoparticles, the method further comprising:
collecting nanoconstructs with the first predetermined size of metallic nanoparticles, and repeating said interacting with a second predetermined size of metallic nanoparticles to produce nanoconstructs with the first and second predetermined sizes of metallic nanoparticles.

9. The method of claim 1, wherein said carrier nanoparticles encapsulated a first payload.

10. The method of claim 9, wherein said metallic nanoparticles are attached to a second payload.

11. The method of claim 10, wherein the metallic nanoparticles are covalently attached to the second payload.

12. The method of claim 10, wherein the first and second payloads comprise first and second drugs.

13. The method of claim 12, wherein the first and second drugs comprise two different drugs.

14. A method of cancer treatment, the method comprising introducing a nanoconstruct produced by the method of claim 10 in vivo, and wherein at least one of the first and second payloads comprises a biomolecule having efficacy for cancer treatment.

15. The method of claim 14, wherein the biomolecule having efficacy for cancer treatment comprises a chemotherapeutic molecule.

16. The method of claim 15, wherein the chemotherapeutic molecule comprises doxorubicin, cis-platin, or gemcitabine.

17. A multidimensional nanoconstruct comprising a protein, thiolated gelatin or polymer nanoparticle and exposed metallic nanoparticles bonded to outer surfaces of the particle.

18. The nanoconstruct of claim 17, wherein the number of metallic nanoparticles comprises a predetermined number within a limited range that is a fraction of the predetermined number on either side (+/−) of the predetermined number.

19. A plurality of nanoconstructs of claim 18, having each having the predetermined number within the limited range of metallic nanoparticles.

20. The nanoconstruct of claim 19, wherein the predetermined number within a limited range is 2-8 metallic nanoparticles and the fraction of the predetermined number is +/−2.

21. The nanoconstruct of claim 19, wherein the predetermined number within a limited range is 18-30 metallic nanoparticles and the fraction of the predetermined number is +/−5.

22. The nanoconstruct of claim 19, wherein the predetermined number within a limited range is 36-60 metallic nanoparticles and the fraction of the predetermined number is +/−15.

23. The nanoconstruct of claim 17, wherein the metallic nanoparticles comprise gold nanoparticles.

24. The nanoconstruct of claim 17, wherein the metallic nanoparticles consist of gold nanoparticles.

25. The nanoconstruct of claim 17, wherein the metallic nanoparticles comprise silver nanoparticles.

26. The nanoconstruct of claim 17, wherein the metallic nanoparticles consist of silver nanoparticles.

27. The nanoconstruct of claim 17, wherein the metallic nanoparticles comprise metal nanoparticles having an affinity towards the sulfhydryl group.

28. The nanoconstruct of claim 18, wherein the nanoconstruct consists essentially of the carrier nanoparticle and the metallic nanoparticles.

29. The nanoconstruct of claim 28, wherein the carrier nanoparticle consists of PEGgylated gelatin.

30. The nanoconstruct of claim 28, wherein the carrier nanoparticle particle consists of HSA (albumin-derived).

31. The nanoconstruct of claim 28, wherein the carrier particle encapsulates a first payload.

32. The nanoconstruct of claim 31, wherein the metallic nanoparticles are attached with a second payload.

33. The nanoconstruct of claim 32, wherein the first payload and the second payload are first and second drugs.
